(19) United States
(12) Patent Application Publication
(10) Pub. No.: US 2026/0008747 A1
(43) Pub. Date: Jan. 8, 2026

(54) SELECTIVE MONOARYLATIONS OF HYDRAZINES

(71) Applicants: Ruhr-Universitaet Bochum, Bochum (DE); UMICORE AG & CO. KG, Hanau-Wolfgang (DE)

(72) Inventors: Lukas J GOOSSEN, Hattingen (DE); Florian PAPP, Bochum (DE); Bingxiang XUE, Bochum (DE); Angelino DOPPIU, Hanau (DE)

(73) Assignees: Ruhr-Universitaet Bochum, Bochum (DE); UMICORE AG & CO. KG, Hanau-Wolfgang (DE)

(21) Appl. No.: 18/773,244

(22) Filed: Jul. 15, 2024

(30) Foreign Application Priority Data
Jul. 5, 2024 (EP) 24186844.7

(51) Int. Cl. C07C 241/02; B01J 31/22; B01J 31/24

(52) U.S. Cl. CPC C07C 241/02; B01J 31/2295; B01J 31/2404; B01J 2231/4283; B01J 2531/004; B01J 2531/824

(57) ABSTRACT

The present invention is directed towards the monoarylation of hydrazines. In particular, a process is described herein to selectively add aryls or hetero aryls to hydrazine via a C—N-cross-coupling reaction using special Pd-phosphine complexes.

The present invention is directed towards the monoarylation of hydrazines. In particular, a process is described herein to selectively add aryls or hetero aryls to hydrazine via a C—N-cross-coupling reaction using special Pd-phosphine complexes.

Palladium catalysis is used in organic synthetic chemistry to produce a wide variety of compounds. The most prominent palladium-catalyzed reactions include C—C bond-forming reactions and C-heteroatom bond-forming reactions, commonly referred to as cross-coupling reactions (EP3845546A). C—N-cross-coupling reactions have already been reported (Ruiz-Castillo, P.; Buchwald, S. L. Applications of Palladium-Catalyzed C—N Cross-Coupling Reactions. Chem. Rev. 2016, 116 (19), 12564-12649.).

E.g., the transition metal-catalyzed monoarylation of ammonia is one of the most attractive syntheses of anilines. However, it is challenging, since the aniline products are more reactive towards a second arylation than ammonia is towards the first. Extensive research has led to the development of Pd-, Ni- and Cu-catalyzed protocols that permit the selective synthesis of anilines from ammonia solutions or ammonium salts. For the Pd-catalyzed reactions, bulky, electron-rich ligands are vital to achieve selective monoarylations (Kim, S.-T.; Kim, S.; Baik, M.-H. How Bulky Ligands Control the Chemoselectivity of Pd-Catalyzed N-Arylation of Ammonia. Chem. Sci. 2020, 11 (4), 1017-1025.). State-of-the-art palladium catalysts such as Buchwald's Pd[Me₃(OMe)XPhos]G3 systems (Surry, D. S.; Buchwald, S. L. Selective Palladium-Catalyzed Arylation of Ammonia: Synthesis of Anilines as Well as Symmetrical and Unsymmetrical Di- and Triarylamines. J. Am. Chem. Soc. 2007, 129 (34), 10354-10355.), Hartwig's JosiPhos (Shen, Q.; Hartwig, J. F. Palladium-Catalyzed Coupling of Ammonia and Lithium Amide with Aryl Halides. J. Am. Chem. Soc. 2006, 128 (31), 10028-10029.), and Stradiotto's MorDalPhos (Lundgren, R. J.; Peters, B. D.; Alsabeh, P. G.; Stradiotto, M. A P,N-Ligand for Palladium-Catalyzed Ammonia Arylation: Coupling of Deactivated Aryl Chlorides, Chemoselective Arylations, and Room Temperature Reactions. Angew Chem Int Ed 2010, 49 (24), 4071-4074) and BippyPhos (Crawford, S. M.; Lavery, C. B.; Stradiotto, M. BippyPhos: A Single Ligand With Unprecedented Scope in the Buchwald-Hartwig Amination of (Hetero)Aryl Chlorides. Chemistry A European J 2013, 19 (49), 16760-16771) catalyst give high yields at low loadings, moderate temperatures and short reaction times.

Compared to the arylation of ammonia, a selective monoarylation of hydrazine is even harder to achieve. The first arylation activates the ipso-nitrogen for a second arylation due to an increased nucleophilicity. Bulky ligands can overrule this, but the second nitrogen atom of a monoarylated hydrazine is sterically as accessible as hydrazine itself. State-of-the art catalysts for monoarylation of hydrazine such as Stradiotto's [Pd(cinnamyl)Cl]₂/MorDalPhos is and Hartwig's Pd[(P(o-tolyl)₃]/JosiPhos system (Wang, J. Y.; Choi, K.; Zuend, S. J.; Borate, K.; Shinde, H.; Goetz, R.; Hartwig, J. F. Cross-Coupling between Hydrazine and Aryl Halides with Hydroxide Base at Low Loadings of Palladium by Rate-Determining Deprotonation of Bound Hydrazine. Angew Chem Int Ed 2021, 60 (1), 399-408) require high temperatures, Buchwald's Pd G1 BrettPhos catalyst an elaborate flow-through protocol (DeAngelis, A.; Wang, D.; Buchwald, S. L. Mild and Rapid Pd-Catalyzed Cross-Coupling with Hydrazine in Continuous Flow: Application to the Synthesis of Functionalized Heterocycles. Angew Chem Int Ed 2013, 52 (12), 3434-3437).

Several of Buchwald's new catalysts for C—N-cross coupling are already commercially available (https://www.sigmaaldrich.com/DE/de/technical-documents/technical-article/chemistry-and-synthesis/cross-coupling/buchwald-g6-precatalysts-oxidative-addition-complexes). They have been proven versatile in lots of C—C- or C—N-cross-coupling reactions. However, in particular for the monoarylation of hydrazine still there exists the need in the art to develop even more active and selective catalysts. Hence, it is an object of the present invention to provide a process and a catalyst for the monoarylation of hydrazines which is superior in activity and selectivity to the processes and catalyst known in the prior art for these kind of reactions.

These and other object being obvious to those skilled in the art are solved by providing a process for the monoarylation of hydrazines according to claim 1. Further preferred embodiments of the inventive process are mentioned in dependent claims 2-8. In claim 9 a new catalyst for this reaction is described. claim 9 focusses on a process for producing the catalyst of claim 10.

By conducting a process for the selective monoarylation of hydrazine hydrate or hydrazine salts of the general formular (I) or (II)

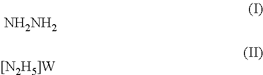

wherein W is a counter anion
which comprises the steps of:
providing an organic polar solvent, which does not interact with the reactants added but dissolves the reactants;
adding thereto as a reactant a hydrazine of formula (I) or (II);
adding a catalyst of the general formula (III) or (IV):

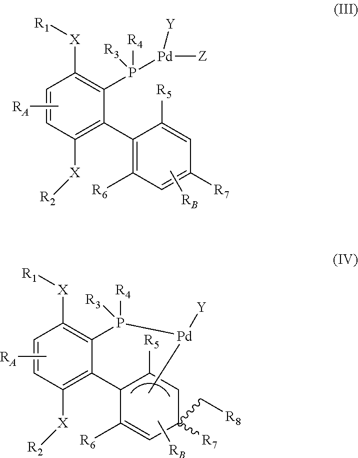

wherein R1, R2 are same or different from each other and being linear or branched alkyl;
R3, R4, are same or different from each other and being branched or cyclic alkyl;
R5, R6, R7 are same or different from each other and being H, linear or branched or cyclic alkyl;
R8 is an alkenyl, aryl or aryl alkenyl group;
R_{A} and R_{B} are if present independently from each other one or more alkyl, cycloalkyl, aryl, heteroaryl, heteroalkyl, aralkyl, hydrogen, halogen, heteroaralkyl, alkoxyl, dialkylaminyl, trialkylsilyl; and
X is O or S, with O being preferred;
Y is an anionic ligand, like halide, tosylate, mesylate, triflate, acetate;
Z is a σ-donor ligand, like aryl or heteroaryl;
   adding as a further reactant an aryl halide or heteroaryl halide to the reaction mixture; and
   adding a base; and
   isolating the arylhydrazine from the reaction mixture, the skilled worker is able to solve the problems addressed above. By using the catalysts mentioned in the process of arylating hydrazines can be performed with an outstandingly high yield. Moreover, the catalysts presented above provide for an extraordinary selectivity in the process of monoarylation hydrazines to an extent which was not foreseeable from the knowledge to date.

In a process according to the invention residues R1 and R2 for the ligands can advantageously and independently be selected from the group consisting of methyl, ethyl, propyl, iso-propyl, butyl, t-butyl, iso-butyl and sec-butyl. In an even more preferred embodiment R2 is methyl, R1 is methyl, t-butyl or iso-propyl. Most preferably, R1 and R2 are methyl groups.

R3, R4 are rather bulky groups which shield the side of the phosphine ligand from any other reaction that can occur. In a preferred embodiment, R3 and R4 are independently from each other tert-butyl, isopropyl, neopentyl, 1-adamantyl, propellane. In a more preferred aspect R3 and R4 are chosen from the group of tert-butyl, or 1-adamantyl.

Likewise R5, R6 and R7 are alkyl substituents also. In a preferred embodiment, R5, R6 and R7 are independently from each other methyl, ethyl, propyl, isopropyl, butyl, iso-butyl and sec-butyl. In a more preferred aspect R5, R6 and R7 are chosen from the group isopropyl, iso-butyl and sec-butyl. Very preferable, R5, R6 and R7 is isopropyl.

R8 can be an aryl, alkenyl or aryl alkenyl moiety. R8 is released from the compound of formula (IV) upon reaction (see scheme 1). In a preferred embodiment R8 can be selected from the group consisting of phenyl, ethenyl, naphthyl, allyl, crotyl or 1-^{t}Bu-indenyl. Very much preferred is if R8 is phenyl ethenyl, naphthyl or vinyl. As will be apparent from the experimental section the position of the residue R8 can be above the attached ring or below. The anomers of formula (IV) can be applied in the inventive reaction with equal selectivity and activity. Hence, if a description of a formula (IV) is given this description always encompasses both possible anomers.

As mentioned Y is a an anionic ligand, like halide, tosylate, triflate, acetate. A halide like Cl or Br are particularly preferred in this connection. Z is a σ-donor ligand like aryl or heteroaryl for instance, or an alkyl or an alkenyl. Z can also be a bidentate ligand in which one part of the ligand is connected to the Palladium via a σ-donor bond as mentioned before and the other part is connected to the Palladium via a heteroatomic substituent. In a very preferred embodiment this heteroatom is a nitrogen. In an extremely preferred aspect the ligand can comprise a biphenyl structure. In an exemplary but utmostly preferred embodiment of formula (III) an aryl is a substituted or unsubstituted biphenyl moiety. E.g., a catalysts like mentioned in formula (V) can also be used in the present reaction.

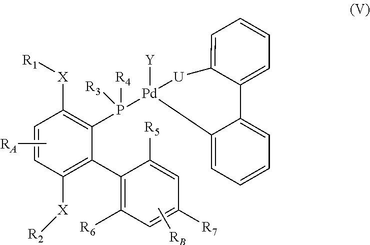

wherein U is selected from the group consisting of NH₂, NHMe. Here the other residues can take the meaning as mentioned before. Compounds of formula (V) are known to those skilled in the art (U.S. Pat. No. 8,889,857B2).

The catalysts (III) (J. Am. Chem. Soc. 2020, 142, 15027-15037, Org. Lett. 2021, 23, 20, 7927-7932) can easily be synthesized according to literature procedures. The catalysts (IV) can be prepared according to the general procedure A mentioned in the experimental section.

The catalysts used for the inventive process are those of formula (III) and (IV) and (V). These catalysts surprisingly provide a selectivity and activity in the present reaction that has been unknown so far. The catalysts are normally used in substoichiometric amounts. Usually, the concentration of these catalysts in the reaction mixture is between 3.3*10⁻⁵-6.7*10⁻³ mol/l, more preferably 1.7*10⁻⁴-6.7*10⁻³ mol/l and most preferably between 3.3*10⁻³-6.7*10⁻³ mol/l.

In the process of the present invention hydrazine or a hydrazine derivative is selectively arylated via a C—N-cross-coupling reaction. The hydrazines that can be used in this reaction are in principle known to those skilled in the art. The hydrazines can be used in form of the hydrazine as such or as a hydrate or salt thereof. Salts to be used are known to the skilled worker. Versatile salts are those having counter anions selected from the group consisting of carboxylates, halides and pseudo halides. Most preferred are anions like OTf⁻, Cl⁻, SO₄²⁻, OAc⁻. These compounds are usually used in a concentration of 0.5-2 mol/l, more preferred 0.5-1 mol/l and very preferred around 0.67 mol/l.

In order to cross-couple the hydrazines or their derivatives with a further reactant an aryl halide has to be added to the reaction mixture. The aryl halide usually is added in concentrations of 0.1-3 mol/l, more preferably 0.33-2 mol/l and most preferably 0.33 mol/l to the reaction mixture. The ratio of reactants has already been addressed above. The ratio between the hydrazines and the aryl halide as the further reactant can be determined by the skilled person. Usually, the molar ratio ranges between 10:1-1:1, more preferred 3:1-1:1 and very preferred 2:1.

As an aryl halide for the C—N-cross-coupling reaction any aryl halide or heteroaryl halide known to the skilled person as being feasible in this reaction can be taken. Normally, the aryl halide consist of an aryl moiety attached to halide. The heteroaryl halide is accordingly. The halide is preferably any of Cl, Br or J. More preferably Br and Cl are addressed here. Most preferably the halide is Cl. The (hetero)aryl moiety can be any (hetero)aryl like e.g. selected from the group consisting of phenyl, naphthyl, pyridyl, pyrazinyl and thienyl. These (hetero)aryls can itself be substituted by further functional groups alkoxy, ketone, formyl, ester, ether, amine, silane and borane groups. In particular, electron-withdrawing substituents can be attached to the aryl moiety. Advantageously, these substituents can be those selected from the group consisting of fluorine, cyano, acyl, formyl, nitro, trifluoromethyl and amide.

The inventive process is conducted quite easily. E.g., in a vessel a kind of organic solvents is provided. The organic solvent should be one which does not interact with the reactants nor with the catalyst in order not to inhibit the reaction or to furnish side product production. On the other hand it should be polar enough to dissolve all the reactants and the catalyst to the necessary extent. The skilled workers can choose the solvent according to his needs. Preferably, the solvents are polar aprotic solvents. More preferably, the solvent is selected from ethereal compounds. Most preferable, the organic solvent is selected from the group consisting of THF, dioxane, dimethoxyethane, methyl t-butyl ether, cyclohexyl methyl ether, 2-methyl-tetrahydrofurane.

Once the solvent is present, the reactants can be added to the solvent. The sequence of addition is not that critical. Finally, a base needs to be added to the reaction mixture. The base can be selected according to the knowledge of the skilled worker. It should be soluble in the reaction mixture to the necessary extent and be basic enough to deprotonate the Pd-coordinated hydrazine. Preferably, the base is selected from the group consisting of KOH, K₂CO₃, K₃PO₄, Cs₂CO₃, NaOtBu, KOtBu, NaOMe, NaOH. Most preferred are KOH and NaOH In this respect.

When all ingredients have been added to the reaction mixture, the monoarylation happens usually in matter of hours at room temperature. In a preferred embodiment the reaction mixture is allowed to be heated though. More preferably, the process of the invention is conducted at a temperature of 10° C. to 80° C., even more preferably at 10-40° C. and most preferably at 25-30° C.

As a final step the products can be isolated from the reaction mixture according to the knowledge of those skilled in the art. Preferably, they can be acidified by concentrated hydrochloride acid to generate the hydrochloride salts and purified by two-phase extractions, precipitation and filtration. Alternatively, they are not isolated but directly converted into heterocycles by reaction with dicarbonyl compounds or ketones for example.

In a further aspect the present invention is directed to a catalyst for the monoarylation of hydrazines of general formula (IV)

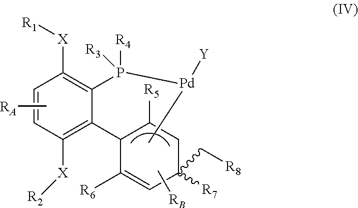

wherein R1, R2 are same or different from each other and being linear or branched alkyl;
R3, R4, are same or different from each other and being branched or cyclic alkyl;
R5, R6, R7 are same or different from each other and being H, linear or branched or cyclic alkyl;
R8 is an alkenyl, aryl or aryl alkenyl group;
R_{A} and R_{B} are if present independently from each other one or more alkyl, cycloalkyl, aryl, heteroaryl, heteroalkyl, aralkyl, hydrogen, halogen, heteroaralkyl, alkoxyl, dialkylaminyl, trialkylsilyl; and
X is O or S, with O being preferred;
Y is a an anionic ligand, like halide, tosylate, mesylate, triflate, acetate.

In a further aspect of the present invention the catalyst of formula (IV) can be produced in a process characterized in that a compound of general formula (VI)

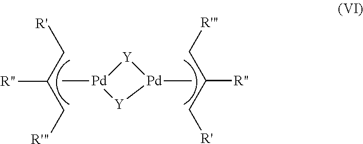

wherein R′, R″, R′″ are independently of each other selected from the group consisting of H, alkyl, aryl, with methyl, ethyl, C3-C8 linear alkyl, phenyl being preferred or R′ and R″ form an aromatic or non-aromatic cyclic ring; like with 1-methyl naphthaline (1-MeNAP); 2-methyl naphthaline (2-MeNAP);
Y is a halide; preferably like Cl or Br;
and a ligand of formula (VII)

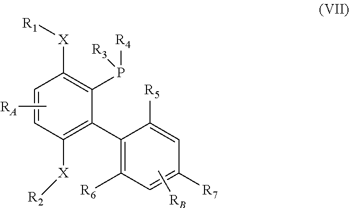

wherein
R1, R2 are same or different from each other and being linear or branched alkyl;
R3, R4, are same or different from each other and being branched or cyclic alkyl;
R5, R6, R7 are same or different from each other and being H, linear or branched or cyclic alkyl;
R_{A} and R_{B} are if present independently from each other one or more alkyl, cycloalkyl, aryl, heteroaryl, heteroalkyl, aralkyl, hydrogen, halogen, heteroaralkyl, alkoxyl, dialkylaminyl, trialkylsilyl;
X is O or S, with O being preferred,
are reacted under conditions sufficient to produce the respective compound. The condition under which the reaction takes place can be selected according the viewpoint of the skilled worker. In general the solvents applied can be selected from the group consisting of ketones, ethers, esters Preferred is acetone, MTBE, THF, ethylacetate. Especially preferred is acetone in this connection.

In general, the temperatures applied can range from 10-100° C., more preferably 15-50° C. and most preferably at around room temperature. The catalyst of formula (IV) can be isolated according to those skilled in the art.

The reaction for forming the catalyst (IV) runs smoothly already under room temperature in a couple of hours, e.g. according to the scheme 1:

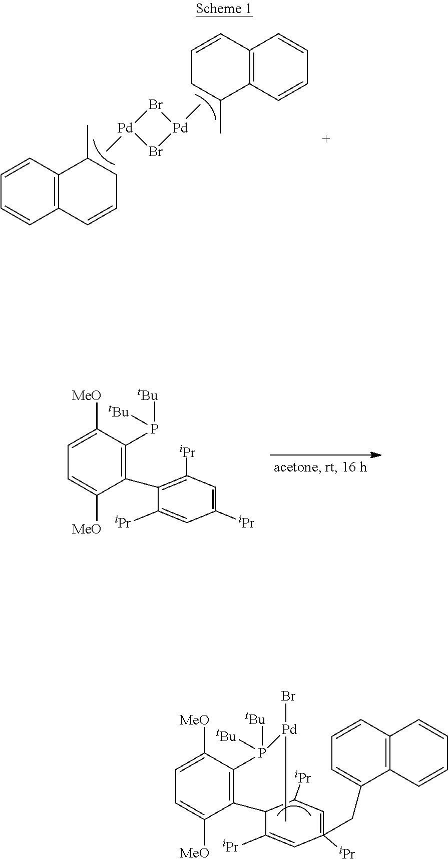

Depending on the initial η-allylic Pd-compound (VI) the final catalyst (IV) can have a differing residue R8, like preferably be an alkenyl or an aryl or an aryl alkenyl moiety, like phenyl, ethenyl, naphthyl, allyl, crotyl or 1-^{t}Bu-indenyl. Very much preferred is if R8 is phenyl ethenyl, crotyl, naphthyl or vinyl. If a molecule as presented under formula (VI) is used R′, R″, R′″ have to be chosen accordingly. E.g., complexes like in scheme 2 have been prepared in situ, albeit they were not isolated in pure form so far.

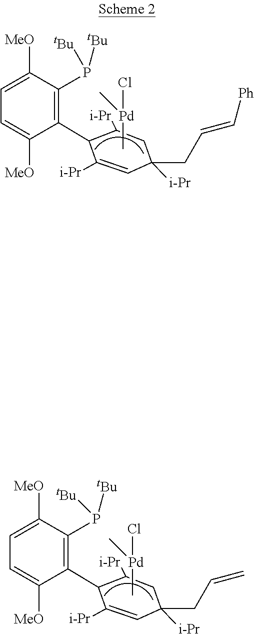

In the process of this invention this catalyst species (IV) shows activities and selectivities for the monoarylation of hydrazines which are paramount. Yields in the region of 99% with nearly no detectable side product could be obtained using these kind of catalysts. A further benefit is that the catalysts are quite easy to produce (e.g. see scheme 1) and manageable so that they are pretty good implementable in a large scale production process. This is all but obvious from the prior art knowledge.

It should be clear that all preferred embodiments mentioned for the catalyst of formula (IV) above also are applicable in respect of the process. In particular, as the catalyst of formula (IV) can exist in two anomeric structures both of them are contemplated when looking at a specific structure.

“Alkyl” refers to a straight-chain or branched saturated hydrocarbon group. In certain embodiments, the alkyl group may have from 1-20 carbon atoms, in certain embodiments from 1-15 carbon atoms, in certain embodiments, 1-8 carbon atoms. The alkyl group may be unsubstituted. Alternatively, the alkyl group may be substituted. Unless otherwise specified, the alkyl group may be attached at any suitable carbon atom and, if substituted, may be substituted at any suitable atom. Typical alkyl groups include but are not limited to methyl, ethyl, n-propyl, iso-propyl, n-butyl, iso-butyl, sec-butyl, tert-butyl, n-pentyl, n-hexyl and the like. “Alkenyl” refer to alkyl having one or more double bonds in it. “Aryl alkenyl” refers to an aryl moiety that is attached to an alkenyl residue.

The term “cycloalkyl” is used to denote a saturated carbocyclic hydrocarbon radical. The cycloalkyl group may have a single ring or multiple condensed rings. In certain embodiments, the cycloalkyl group may have from 3-15 carbon atoms, in certain embodiments, from 3-10 carbon atoms, in certain embodiments, from 3-8 carbon atoms. The cycloalkyl group may be unsubstituted. Alternatively, the cycloalkyl group may be substituted. Unless other specified, the cycloalkyl group may be attached at any suitable carbon atom and, if substituted, may be substituted at any suitable atom. Typical cycloalkyl groups include but are not limited to cyclopropyl, cyclobutyl, cyclopentyl, cyclohexyl and the like.

“Aryl” refers to an aromatic carbocyclic group. The aryl group may have a single ring or multiple condensed rings. In certain embodiments, the aryl group can have from 6-20 carbon atoms, in certain embodiments from 6-15 carbon atoms, in certain embodiments, 6-12 carbon atoms. The aryl group may be unsubstituted. Alternatively, the aryl group may be substituted. Unless otherwise specified, the aryl group may be attached at any suitable carbon atom and, if substituted, may be substituted at any suitable atom. Examples of aryl groups include, but are not limited to, phenyl, naphthyl, anthracenyl and the like.

“Heteroalkyl” refers to a straight-chain or branched saturated hydrocarbon group wherein one or more carbon atoms are independently replaced with one or more heteroatoms (e.g. nitrogen, oxygen, phosphorus and/or sulfur atoms). The heteroalkyl group may be unsubstituted. Alternatively, the heteroalkyl group may be substituted. Unless otherwise specified, the heteroalkyl group may be attached at any suitable atom and, if substituted, may be substituted at any suitable atom. Examples of heteroalkyl groups include but are not limited to ethers, thioethers, primary amines, secondary amines, tertiary amines and the like.

“Heterocycloalkyl” refers to a saturated cyclic hydrocarbon group wherein one or more carbon atoms are independently replaced with one or more heteroatoms (e.g. nitrogen, oxygen, phosphorus and/or sulfur atoms). The heterocycloalkyl group may be unsubstituted. Alternatively, the heterocycloalkyl group may be substituted. Unless otherwise specified, the heterocycloalkyl group may be attached at any suitable atom and, if substituted, may be substituted at any suitable atom. Examples of heterocycloalkyl groups include but are not limited to epoxide, morpholinyl, piperadinyl, piperazinyl, thirranyl, pyrrolidinyl, pyrazolidinyl, imidazolidinyl, thiazolidinyl, thiomorpholinyl and the like.

“Heteroaryl” refers to an aromatic carbocyclic group wherein one or more carbon atoms are independently replaced with one or more heteroatoms (e.g. nitrogen, oxygen, phosphorus and/or sulfur atoms). The heteroaryl group may be unsubstituted. Alternatively, the heteroaryl group may be substituted. Unless otherwise specified, the heteroaryl group may be attached at any suitable atom and, if substituted, may be substituted at any suitable atom. Examples of heteroaryl groups include but are not limited to thienyl, furanyl, pyrrolyl, imidazolyl, pyrazolyl, thiazolyl, isothiazolyl, oxazolyl, isoxazolyl, triazolyl, thiadiazolyl, thiophenyl, oxadiazolyl, pyridinyl, pyrimidyl, benzoxazolyl, benzthiazolyl, benzimidazolyl, indolyl, quinolinyl and the like.

“Arylalkyl” refers to an optionally substituted group of the formula aryl-alkyl-, where aryl and alkyl are as defined above.

It should be noted that the possibilities for the residues whether advantageous, preferred, or not preferred, mentioned in the present text are also applicable for the residues in the other formulas having same abbreviation for the residues.

In a nutshell, it has to be noticed that the process of the invention and the catalysts of formula (IV) have beneficial properties within the field of monoarylation of hydrazines. The catalysts are easy to produce, the condition under which the process runs are mild and the yields and selectivities for the formation of monoarylated products are superb. This is very surprising based on the prior art known to the skilled person.

### FIGURES

FIG. 1: ESI-MS spectrum of reaction mixture of [Pd(cinnamyl)(^{t}BuBrettPhos)Cl] with 0.01 mmol [Pd(cinnamyl)Cl]₂, 2.0 equiv. ^{t}BuBrettPhos in 2 mL THF for 30 min at 80° C.

FIG. 2: ESI-MS spectrum of isolated [Pd(allyl)(^{t}BuBrettPhos)Cl]following general procedure A in THF with 0.25 mmol [Pd(allyl)Cl]₂.

FIG. 3: Comparison of ¹H NMR spectra of [Pd(1-MeNAP)(tBuBrettPhos)Br](bottom), ^{t}BuBrettPhos (middle), and [Pd(1-MeNAP)Br]₂ (top). ¹H NMR spectra were recorded at 400 MHz in CD₂Cl₂ or DMSO-d₆ (for [Pd(1-MeNAP)Br]₂ due to low solubility in DCM).

FIG. 4: Solid state structure of Pd(2-MeNAP)Br-^{t}BuBrettPhos complex. Hydrogen atoms are omitted for clarity. The thermal ellipsoids are drawn at the 50% probability level. Disorder of 2-methylnaphthyl group was omitted for clarity.

FIG. 5: Solid state structure of Pd(1-MeNAP)Br-^{t}BuBrettPhos complex. Hydrogen atoms are omitted for clarity. The thermal ellipsoids are drawn at the 50% probability level.

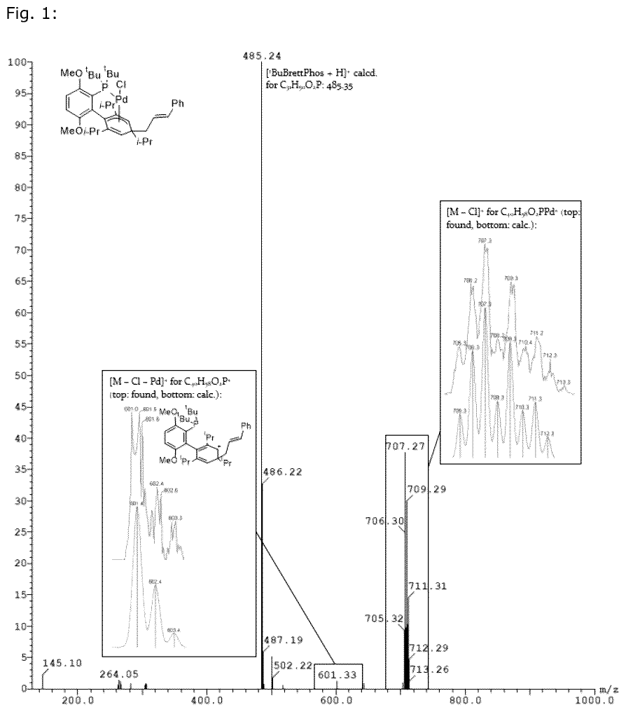

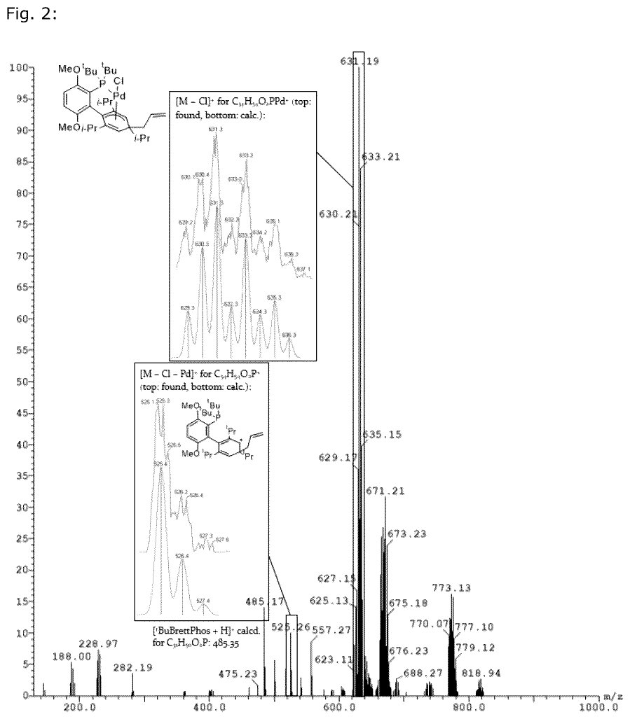

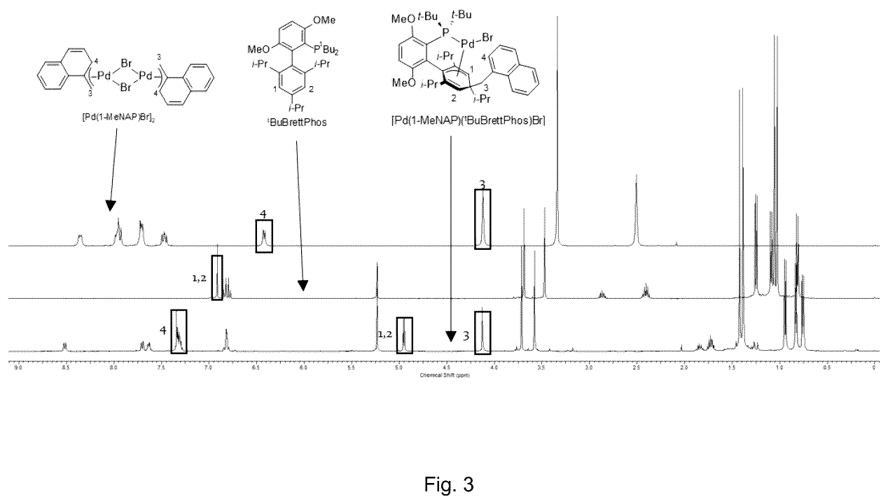

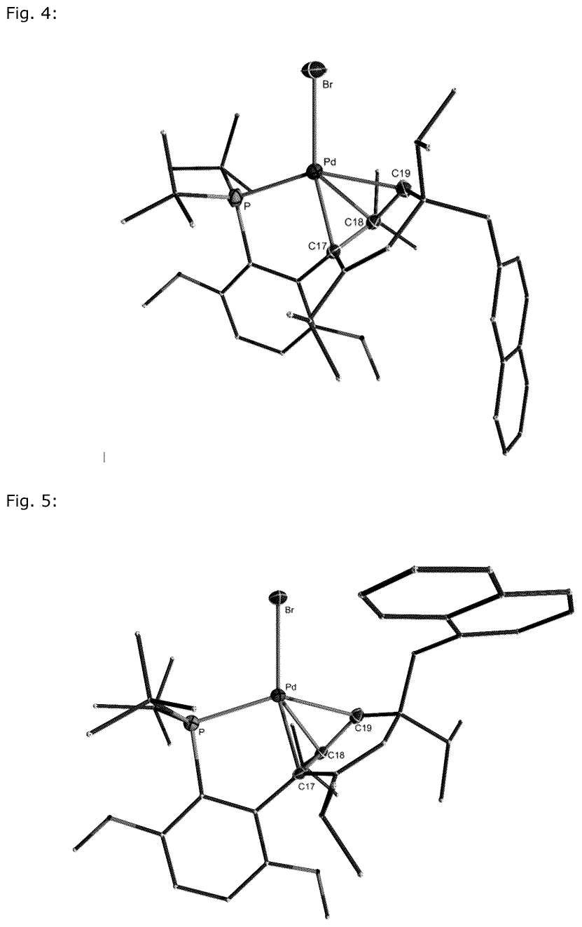

### EXPERIMENTAL REPORT

### I. General

All reactions were performed in oven-dried glassware containing a Teflon-coated stirring bar and dry septum under nitrogen atmosphere. Optimization reactions were monitored by ¹⁹F NMR analysis using 1, 4-difluorobenzene as internal standard.

Characterization of the compounds was done by:

¹H, ¹³C{1H}, and ³¹P NMR spectra were recorded on an Avance-III-300 or Avance-III-400 spectrometer at 25° C. if not stated otherwise. ¹⁹F NMR spectra were recorded on Spinsolve Benchtop NMR (MAGRITEK) spectrometers at 25° C. All values of the chemical shift are in ppm regarding the 5-scale. To display multiplicities and signal forms correctly the following abbreviations were used: s=singlet, d=doublet, t=triplet, q=quartet, spt=septet, m=multiplet, dd=doublet of doublet, dq=doublet of quartet, dspt=doublet of septet, br=broad signal. Column chromatography was performed on a CombiFlash Companion (Isco) and a Pure C-815 Flash (Buchi) using Reveleris packed columns (12 g or 40 g). Mass spectrometric data (EI) were acquired on a GC-MS Agilent 5977B MSD. Mass spectra (ESI) of Pd complexes were recorded via direct injection using acetonitrile/water (0.1% formic acid) as eluent. Samples were prepared by dissolving the complex in acetonitrile (ca. 1 mg/mL) and filtration through a PTFE syringe filter. The evaluation and calculation of MS spectra were performed with the MassLynx software HRMS analyses were acquired using a GC-MS system consisting of an Agilent 7250 GC/Q-TOF, in which ionization was achieved by EI. Infrared spectra were recorded on Bruker Vertex 70 Spectrometer with Universal ATR Sampling Accessory. Melting points were measured on a Mettler Toledo MP70. Elemental analysis was performed on a varioMICRO CHNS.

Commercial substrates were used as received unless otherwise stated. Ammonium triflate was washed with ether, dried in vacuo, and stored in the glovebox. Solvents were purchased (puriss p.A.) from commercial suppliers and dried by standard procedures (Armarego, W. L. F.; Chai, C. L. L. Purification of Laboratory Chemicals, 5th ed.; Butterworth-Heinemann: Amsterdam; Boston, 2003). All solvents and liquid reactants were degassed by Argon purge prior to use. [Pd(1-MeNAP)Br]₂ as well as other Pd sources were donated by Umicore (EP4267591A). KOH pellets were imported into a glovebox and pulverized into a fine powder with a mortar and pestle. [N₂H₅][OTf] was synthesized by previously reported procedure (Mock, M. T.; Chen, S.; O'Hagan, M.; Rousseau, R.; Dougherty, W. G.; Kassel, W. S.; Bullock, R. M. Dinitrogen Reduction by a Chromium(0) Complex Supported by a 16-Membered Phosphorus Macrocycle. J. Am. Chem. Soc. 2013, 135 (31), 11493-11496).

### II. Production of the Catalysts Tested

### General Preparation of Pd Pre-Catalysts—Procedure A

### Catalysts of Formula (IV)

[Pd(MeNAP)Br]₂ (EP4267591A) (0.125 or 0.25 mmol) and ligand (2.0 equiv.) were weighed in an oven-dry vial. After addition of 5 or 10 mL THF or acetone, the reaction mixture was stirred at room temperature overnight. 90% of the solvent was evaporated and 5 or 10 mL pentane was added. The mixture was stored at −20° C. over night to crystallize the product. The mother liquor was decanted and the remaining solid was washed with pentane (3×5 mL) and dried under high vacuum to afford the Pd complex.

### Catalysts of Formula (III) and (V):

The catalysts are commercially available under https://www.sigmaaldrich.com/DE/en/technical-documents/technical-article/chemistry-and-synthesis/cross-coupling/buchwald-g6-precatalysts-oxidative-addition-complexes or can be prepared as mentioned in: King, R.; Senecal, T. D.; Shu, W.; Buchwald, S. L. A General, Practical Palladium-Catalyzed Cyanation of (Hetero)Aryl Chlorides and Bromides. Angew. Chem. Int. Ed. 2013, 52 (38), 10035-10039.

### III. Results on Generation of Pd Catalysts

### 2-di-tert-butylphosphino(2′,4′,6′-triisopropyl-3,6-dimethoxy-4′-(naphthalen-1-ylmethyl)-[1,1′-biphenyl])bromo-palladium(II) [Pd(1-MeNAP)(tBuBrettPhos)Br]

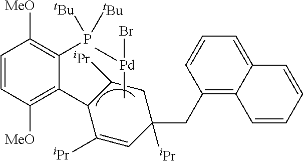

Following general procedure A with [Pd(1-MeNAP)Br]₂ (81.9 mg, 0.125 mmol) and ^{t}BuBrettPhos (122 mg, 0.25 mmol) in acetone, the title compound was obtained as yellow solid (169 mg, 0.208 mmol, 83%).

¹H NMR (400 MHz, CD₂Cl₂): δ=8.61-8.59 (m, 1H), 7.80-7.70 (m, 2H), 7.44-7.36 (m, 4H), 6.93-6.87 (m, 2H), 5.04 (s, 1H), 5.03 (s, 1H), 4.21 (s, 2H), 3.80 (s, 3H), 3.66 (s, 3H), 1.93 (sept., J=6.8 Hz, 1H), 1.86-1.76 (m, 2H), 1.49 (d, J=14.9 Hz, 18H), 1.03 (d, J=6.9 Hz, 6H), 0.92 (d, J=6.6 Hz, 6H), 0.84 (d, J=6.7 Hz, 6H) ppm.

¹³C NMR (75 MHz, CD₂Cl₂): δ=155.7 (d, J=2.8 Hz), 151.2 (d, J=22.9 Hz), 140.0 (d, J=22.9 Hz), 136.3, 134.5 (d, J=6.0 Hz), 134.3 (d, J=4.5 Hz), 133.8 (d, J=15.5 Hz), 129.3, 128.4 (d, J=3.0 Hz), 127.2, 126.0, 125.6, 125.4, 113.9 (d, J=1.7 Hz), 112.3 (d, J=18.1 Hz), 111.4 (d, J=3.9 Hz), 92.3 (d, J=9.2 Hz), 55.0 (d, J=3.0 Hz), 54.8, 54.5, 50.6 (d, J=8.3 Hz), 43.8 (d, J=9.1 Hz), 38.4 (d, J=7.2 Hz), 32.2, 32.1 (d, J=7.7 Hz), 31.9, 31.0, 25.4, 23.4, 17.8 ppm.

³¹P NMR (162 MHz, CD₂Cl₂): δ=94.3, 84.3 ppm.

MS (ESI-TOF): m/z (%)=772.38 (2), 731.22 (100).

EA: Calc. for C₄₂H₅₈O₂PBrPd: C, 62.11%; H, 7.20%; N, 0.00%; Found: C, 62.01%; H, 6.84%; N, 0.00%.

### 2-di-tert-butylphosphino(2′,4′,6′-triisopropyl-3,6-dimethoxy-4′-(naphthalen-2-ylmethyl)-[1,1′-biphenyl])bromo-palladium(II) [Pd(2-MeNAP)(tBuBrettPhos)Br]

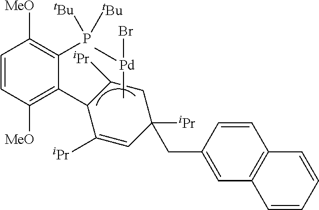

Following general procedure A with [Pd(2-MeNAP)Br]₂ (166 mg, 0.25 mmol) and ^{t}BuBrettPhos (253 mg, 0.50 mmol) in THF, the title compound was obtained as yellow solid (287 mg, 0.353 mmol, 71%).

¹H NMR (400 MHz, CD₂Cl₂): δ=7.80-7.78 (m, 1H), 7.75-7.73 (m, 1H), 7.68 (d, J=8.3 Hz, 1H), 7.62-7.59 (m, 1H), 7.45-7.36 (m, 2H), 7.30 (dt, J=8.6, 0.8 Hz, 1H), 6.81-6.72 (m, 2H), 5.63 (s, 1H), 5.61 (s, 1H), 3.73 (s, 3H), 3.02 (s, 3H), 2.98 (s, 2H), 2.43 (sept, J=6.8 Hz, 1H), 1.68-1.59 (m, 2H), 1.35 (d, J=14.9 Hz, 18H), 1.25 (d, J=6.9 Hz, 6H), 1.06 (d, J=6.7 Hz, 6H), 0.66 (d, J=6.6 Hz, 6H) ppm.

¹³C NMR (75 MHz, CD₂Cl₂): δ=155.3 (d, J=2.8 Hz), 151.1 (d, J=23.9 Hz), 140.1 (d, J=24.2 Hz), 137.0, 134.9 (d, J=6.0 Hz), 133.6, 133.6 (d, J=16.6 Hz), 132.7, 130.6, 129.7, 128.1, 127.9, 127.2, 126.1, 125.5, 113.7 (d, J=1.7 Hz), 113.5, 113.3, 111.1 (d, J=3.3 Hz), 100.6, 89.7 (d, J=10.0 Hz), 54.6, 53.9, 50.0 (d, J=7.5 Hz), 43.9 (d, J=10.6 Hz), 41.0 (d, J=18.9 Hz), 38.3 (d, J=7.2 Hz), 31.9 (d, J=7.2 Hz), 30.8, 24.4 (d, J=6.0 Hz), 18.9 ppm.

³¹P NMR (162 MHz, CD₂Cl₂): δ=85.2 ppm.

MS (ESI-TOF): m/z (%)=772.42 (13), 731.29 (100), 590.14(1), 485.24 (2).

### 2-diadamantylphosphino(2′,4′,6′-triisopropyl-3,6-dimethoxy-4′-(naphthalen-1-ylmethyl)-[1,1′-biphenyl])bromo-palladium(II) [Pd(1-MeNAP)(AdBrettPhos)Br]

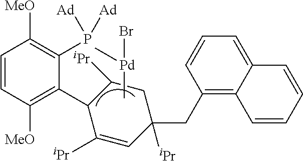

Following general procedure A with [Pd(1-MeNAP)Br]₂ (81.9 mg, 0.125 mmol) and AdBrettPhos (169 mg, 0.25 mmol) in acetone, the title compound was obtained as yellow solid (195 mg, 0.201 mmol, 81%).

¹H NMR (400 MHz, CD₂Cl₂): δ=8.58-8.55 (m, 1H), 7.81-7.71 (m, 2H), 7.46-7.33 (m, 4H), 6.90 (d, J=1.2 Hz, 2H), 5.06 (s, 1H), 5.04 (s, 1H), 4.22 (s, 2H), 3.84 (s, 3H), 3.65 (s, 3H), 2.29 (br. s, 12H), 2.01 (br. s, 6H), 1.85-1.71 (m, 15H), 1.03 (d, J=6.9 Hz, 6H), 0.93-0.91 (m, 12H) ppm.

¹³C NMR (75 MHz, CD₂Cl₂): δ=155.7 (d, J=2.2 Hz), 151.2 (d, J=23.4 Hz), 140.5 (d, J=23.8 Hz), 136.3, 134.4 (d, J=1.5 Hz), 134.3, 132.6 (d, J=13.8 Hz), 129.4, 128.4 (d, J=6.0 Hz), 127.2, 126.0, 125.7, 125.3, 113.6 (d, J=1.7 Hz), 112.3 (d, J=21.1 Hz), 111.1 (d, J=3.3 Hz), 92.2 (d, J=8.8 Hz), 55.0 (d, J=3.3 Hz), 54.8, 50.7 (d, J=8.3 Hz), 43.7, 43.5 (d, J=5.3 Hz), 42.3 (d, J=3.8 Hz), 37.2 (d, J=1.1 Hz), 32.0 (d, J=18.1 Hz), 29.9 (d, J=9.1 Hz), 25.4, 23.6, 17.8 ppm.

³¹P NMR (162 MHz, CD₂Cl₂): δ=100.3, 86.2 ppm. MS (ESI-TOF): m/z (%)=887.51 (32).

EA: Calc. for C₅₄H₇₀O₂PBrPd: C, 66.97%; H, 7.29%; N, 0.00%; Found: C, 67.09%; H, 6.94%; N, 0.00%.

### 2-diadamantylphosphino(2′,4′,6′-triisopropyl-3,6-dimethoxy-4′-(naphthalen-2-ylmethyl)-[1,1′-biphenyl])bromo-palladium(II) [Pd(2-MeNAP)(AdBrettPhos)Br]

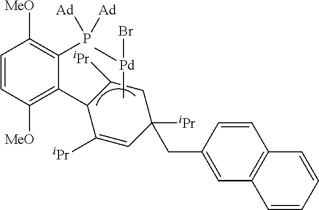

Following general procedure A with [Pd(2-MeNAP)Br]₂ (165 mg, 0.25 mmol) and AdBrettPhos (358 mg, 0.50 mmol) in THF, the title compound was obtained as yellow solid (375 mg, 0.387 mmol, 77%).

¹H NMR (400 MHz, CD₂Cl₂): δ=7.80-7.73 (m, 2H), 7.69 (d, J=8.2 Hz, 1H), 7.62-7.59 (m, 1H), 7.45-7.36 (m, 2H), 7.30 (dd, J=8.4, 0.9 Hz, 1H), 6.82-6.71 (m, 2H), 5.61 (s, 1H), 5.59 (s, 1H), 3.77 (s, 3H), 3.03 (s, 3H), 2.98 (s, 2H), 2.51-2.38 (m, 1H), 2.20-2.11 (m, 12H), 1.89 (s, 6H), 1.69-1.59 (m, 14H), 1.27 (d, J=6.7 Hz, 6H), 1.08 (d, J=6.7 Hz, 6H), 0.65 (d, J=6.6 Hz, 6H) ppm.

¹³C NMR (75 MHz, CD₂Cl₂): δ=155.2 (d, J=2.2 Hz), 151.2 (d, J=23.6 Hz), 140.5 (d, J=23.6 Hz), 137.1, 135.1 (d, J=5.3 Hz), 133.6, 132.7, 132.3 (d, J=15.5 Hz), 130.6, 129.7, 128.1, 127.9, 127.2, 126.0, 125.5, 113.5 (d, J=1.9 Hz), 113.5, 113.3, 110.9 (d, J=3.9 Hz), 89.1 (d, J=9.4 Hz), 54.6, 53.8, 50.0 (d, J=8.3 Hz), 43.6 (d, J=9.8 Hz), 43.2 (d, J=5.5 Hz), 42.1 (d, J=3.3 Hz), 41.2 (d, J=18.9 Hz), 37.1 (d, J=1.1 Hz), 30.7, 29.8 (d, J=9.1 Hz), 24.4 (d, J=6.0 Hz), 19.0 ppm.

³¹P NMR (162 MHz, CD₂Cl₂): δ=86.4 ppm.

MS (ESI-TOF): m/z (%)=928.44 (22), 887.44 (100), 827.22 (7), 746.41 (2) 641.35 (11).

### IV. General Procedure for the Arylation of Hydrazinium Triflate

An oven-dried vial was charged with [Pd(1-MeNAP)(^{t}BuBrettPhos)Br](8.1 mg, 0.01 mmol, 1.0 mol %) and aryl chloride (1.0 mmol, 1.0 equiv., if solid), then KOH (253 mg, 4.5 mmol, 4.5 equiv.), [N₂H₅][OTf](364 mg, 2.0 mmol, 2.0 equiv.), aryl chloride (1.0 mmol, 1.0 equiv., if liquid) and 3 mL 1,4-Dioxane were added in the glovebox and the vial was closed with a septum cap. The resulting mixture was stirred at 25° C. for 16 h. Afterwards, acetylacetone (929 μL, 9 mmol, 9.0 equiv) was added to the reaction and the mixture was stirred at 100° C. for 6 h. The reaction was then cooled to room temperature, diluted with EtOAc (30 mL), and washed with saturated NaHCO₃ (30 mL), water (30 mL) and brine (30 mL). The organic phase was dried over MgSO₄ and purified by flash column chromatography (SiO₂, cyclohexane/ethyl acetate) to yield the products. Alternatively, the reaction mixture was diluted with diethyl ether after the reaction time of 16 h, and washed with saturated Na₂CO₃ (30 mL), water (30 mL) and brine (30 mL). And then the organic layer was separated and acidified to pH=3-4 by adding 37% HCl. The precipitate was filtered, washed with diethyl ether and dried under the vacuum to afford the corresponding aryl hydrazine hydrochloride salts.

### V. Isolating and X-Ray of the Crystals of Catalyst (IV)

Single crystals of Pd(2-MeNAP)Br-^{t}BuBrettPhos were grown by slow diffusion of n-hexane into a saturated solution of the complex in toluene. A crystal was taken up in perfluorinated oil and mounted onto a fiber loop on a Rigaku Oxford diffraction XtaLAB SuperNova diffractometer equipped with an Atlas CCD detector. The crystal was kept at 110.00(10) K during data collection. The obtained diffraction data was analyzed using CrysAlisPro software package. Using Olex2, the structure was solved with the ShelXT structure solution program using Intrinsic Phasing and refined with the ShelXL refinement package using Least Squares minimization (0. V. Dolomanov, L. J. Bourhis, R. J. Gildea, J. A. K. Howard, H. Puschmann, J. Appl. Crystallogr. 2009, 42, 339-341; G. M. Sheldrick, Acta Crystallogr. Sect. Found. Adv. 2015, 71, 3-8; G. M. Sheldrick, Acta Crystallogr. Sect. C Struct. Chem. 2015, 71, 3-8; P. Van Der Sluis, A. L. Spek, Acta Crystallogr A Found Crystallogr 1990, 46, 194-201).

Crystal data and structure refinement for Pd(2-MeNAP)Br-^{t}BuBrettPhos complex:

| Identification code | paf1947b_auto_a.cif | |
|---|---|---|
| Empirical formula | C37.33 H51.56 Br0.89 O1.78 P0.89 Pd0.89 | |
| Formula weight | 721.92 | |
| Temperature | 110(2) K | |
| Wavelength λ | 1.54184 Å (Cu Kα) | |
| Crystal system | monoclinic | |
| Space group | C 2/c (no. 15) | |
| Unit cell dimensions | a = 21.0683(3) Å | α = 90° |
| | b = 11.5672(2) Å | β = 91.5110(10)° |
| | c = 31.8109(5) Å | γ = 90° |

| | |
|---|---|
| Volume | 7749.7(2) Å³ |
| Z | 9 |
| Density ρ(calculated) | 1.392 g/cm³ |
| Absorption coefficient μ | 5.709 mm⁻¹ |
| F(000) | 3376 |
| Crystal size | 0.344 × 0.136 × 0.098 mm³ |
| Theta range for data collection | 2.779 to 76.405° |
| Index ranges | −24 <= h <= 26, −7 <= k <= 14, −39 <= l <= 38 |
| Reflections collected | 25989 |
| Independent reflections | 7566 [Rᵢₙₜ = 0.0280, R_{sigma} = 0.0260] |
| Completeness to theta = 67.684° | 99.3% |
| Absorption correction | multi-scan |
| Max. and min. transmission | 1.00000 and 0.44247 |
| Refinement method | full-matrix least-squares on F² |
| Data/restraints/parameters | 7566/0/457 |
| Goodness-of-fit on F² | 1.038 |
| Final R indices [I > 2 σ(I)] | R₁ = 0.0308, wR₂ = 0.0767 |
| R indices (all data) | R₁ = 0.0331, wR₂ = 0.0779 |
| Extinction coefficient | n/a |
| Largest diff. peak and hole | 1.311 and −0.786e⁻Å⁻³ |

and

Single crystals of Pd(1-MeNAP)Br-^{t}BuBrettPhos were grown by slow diffusion of n-hexane into a saturated solution of the complex in toluene. A crystal was taken up in perfluorinated oil and mounted onto a fiber loop on a Rigaku Oxford diffraction XtaLAB SuperNova diffractometer equipped with an Atlas CCD detector. The crystal was kept at 110.00(10) K during data collection. The obtained diffraction data was analyzed using CrysAlisPro software package. Using Olex2, the structure was solved with the ShelXT structure solution program using Intrinsic Phasing and refined with the ShelXL refinement package using Least Squares minimization (O. V. Dolomanov, L. J. Bourhis, R. J. Gildea, J. A. K. Howard, H. Puschmann, J. Appl. Crystallogr. 2009, 42, 339-341; G. M. Sheldrick, Acta Crystallogr. Sect. Found. Adv. 2015, 71, 3-8; G. M. Sheldrick, Acta Crystallogr. Sect. C Struct. Chem. 2015, 71, 3-8; P. Van Der Sluis, A. L. Spek, Acta Crystallogr A Found Crystallogr 1990, 46, 194-201).

Crystal data and structure refinement for Pd(1-MeNAP)Br-^{t}BuBrettPhos complex.

| Identification code | xub2255_auto_a.cif | |
|---|---|---|
| Empirical formula | C33.60 H46.40 Br0.8 O1.60 P0.80 Pd0.80 | |
| Formula weight | 649.73 | |
| Temperature | 110(2) K | |
| Wavelength λ | 1.54184 Å (Cu Kα) | |
| Crystal system | monoclinic | |
| Space group | P 21/c (no. 14) | |
| Unit cell dimensions | a = 13.5515(2) Å | α = 90° |
| | b = 12.38630(10) Å | β = 97.4060(10)° |
| | c = 22.8312(3) Å | γ = 90° |

| | |
|---|---|
| Volume | 3800.31(8) Å³ |
| Z | 5 |
| Density ρ(calculated) | 1.419 g/cm³ |
| Absorption coefficient μ | 5.821 mm⁻¹ |
| F(000) | 1688 |
| Crystal size | 0.240 × 0.204 × 0.131 mm³ |
| Theta range for data collection | 3.289 to 77.056° |
| Index ranges | −16 <= h <= 17, −15 <= k <= 6, −27 <= l <= 28 |
| Reflections collected | 27573 |
| Independent reflections | 7616 [Rᵢₙₜ = 0.0425, R_{sigma} = 0.0370] |
| Completeness to theta = 67.684° | 100% |
| Absorption correction | multi-scan |
| Max. and min. transmission | 1.00000 and 0.55798 |
| Refinement method | full-matrix least-squares on F² |
| Data/restraints/parameters | 7616/0/438 |
| Goodness-of-fit on F² | 1.043 |
| Final R indices [I > 2 σ(I)] | R₁ = 0.0298, wR₂ = 0.0747 |
| R indices (all data) | R₁ = 0.0326, wR₂ = 0.0763 |
| Extinction coefficient | n/a |
| Largest diff. peak and hole | 0.651 and −0.802e⁻Å⁻³ |

### VI. Results on Hydrazine Coupling of Aryl Chloride

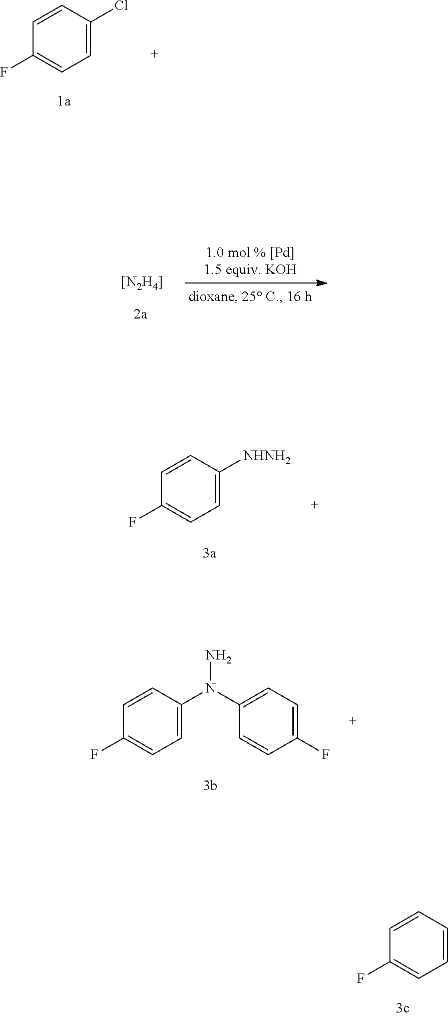

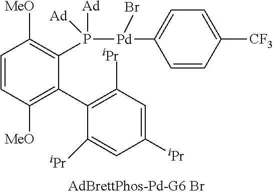

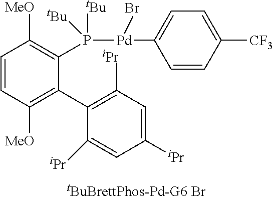

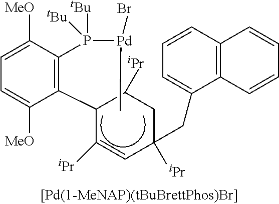

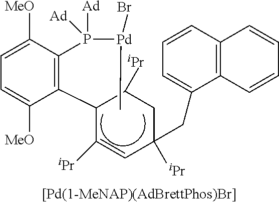

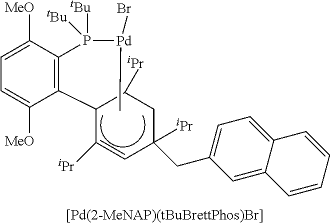

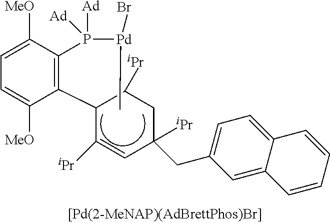

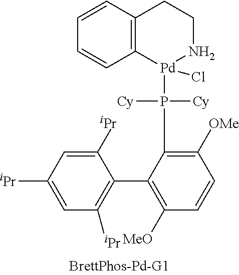

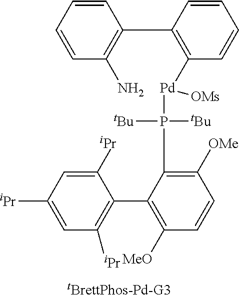

| | | | Ratio | | | |
|---|---|---|---|---|---|---|
| Entry | Catalyst | 2a [N₂H₄] | (1a/2a) | 3a [%] | 3b [%] | 3c [%] |
| 1 | Pd[P(o-tolyl)₃]₂/JosiPhos | N₂H₄•H₂O | 1:1 | 1 | <1 | <1 |
| 2ª | [Pd(cinnamyl)Cl]₂/MorDalPhos | ″ | ″ | 14 | 3 | 5 |
| 3 | [BrettPhos-Pd-G1] | ″ | ″ | 18 | 2 | 8 |
| 4 | Pd(OAc)₂/^{t}BuBrettPhos | ″ | ″ | <1 | <1 | <1 |
| 5 | Pd₂dba₃/^{t}BuBrettPhos | ″ | ″ | 14 | <1 | 32 |
| 6 | [Pd(allyl)Cl]₂/^{t}BuBrettPhos | ″ | ″ | 3 | <1 | 4 |
| 7 | [Pd(cinnamyl)Cl]₂/^{t}BuBrettPhos | ″ | ″ | 13 | <1 | 9 |
| 8 | [Pd(1-MeNAP)Br]₂/^{t}BuBrettPhos | ″ | ″ | 14 | <1 | 16 |
| 9 | [^{t}BuBrettPhos-Pd-G3] | ″ | ″ | 66 | <1 | <1 |
| 10 | [^{t}BuBrettPhos-Pd-G6 Br] | ″ | ″ | 84 | <1 | 2 |
| 11 | [AdBrettPhos-Pd-G6 Br] | ″ | ″ | 51 | <1 | 2 |
| 12 | [Pd(1-MeNAP)(^{t}BuBrettPhos)Br] | ″ | ″ | 77 | <1 | 2 |
| 13^{b} | [^{t}BuBrettPhos-Pd-G3] | [N₂H₅][OTf] | 1:2 | 87 | <1 | <1 |
| 14^{b} | [^{t}BuBrettPhos-Pd-G6 Br] | ″ | ″ | 89 | <1 | 4 |
| 15^{b} | [Pd(1-MeNAP)(^{t}BuBrettPhos)Br] | ″ | ″ | 98 | <1 | <1 |

Conditions: 1.0 mmol 1a, 1.0 mol% [Pd], 1.0 mol% ligand, 1.5 equiv. KOH, 1.5 mL dioxane, 25° C., 16 h. ¹⁹F NMR yields with 1,4-difluorobenzene as internal standard.
^{a}[Pd]/ligand = 1:1.5
^{b}4.5 equiv. KOH, 3 mL dioxane

### Additional Screening Results

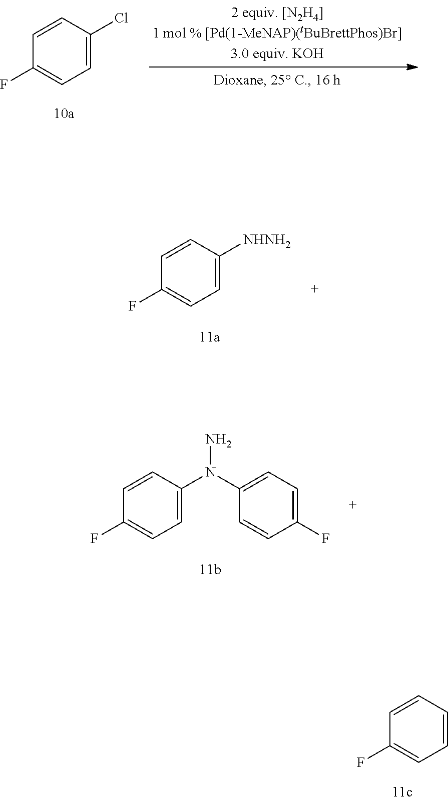

| entry | [N₂H₄] | 11a [%] | 11b [%] | 11c [%] |
|---|---|---|---|---|
| 1 | N₂H₄•H₂O | 95 | <1 | <1 |
| 2^{b} | [N₂H₅][OAc] | 82 | ″ | 3 |
| 3^{b} | [N₂H₅][Cl] | 95 | ″ | <1 |
| 4^{b} | [N₂H₅][Br] | 34 | ″ | ″ |
| 5^{b} | [N₂H₅] | 93 | ″ | ″ |
| | 1/2[SO₄] | | | |
| 6^{b} | [N₂H₅][OTf] | 96 | ″ | ″ |
| 7^{bc} | [N₂H₅][OTf] | 98 | ″ | ″ |

^{a}Conditions: 1.0 mmol 10a, 2.0 equiv. [N₂H₄], 1.0 mol% [Pd(1-MeNAP)(^{t}BuBrettPhos)Br], 3.0 equiv. KOH, 1.5 mL Dioxane, 25° C., 16 h.
^{b}4.5 equiv. KOH.
^{c}3 mL Dioxane. Yields determined by ¹⁹F NMR using 1,4-difluorobenzene as internal standard.

1. Process for the selective monoarylation of hydrazine hydrate or hydrazine salts of the general formular (I) or (II): 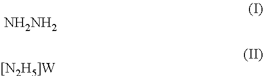
wherein W is a counter anion which comprises the steps of:
providing an organic polar solvent, which does not interact with the reactants added but dissolves the reactants;
adding thereto as a reactant a hydrazine of formula (I) or (II);
adding a catalyst of the general formula (III) or (IV): 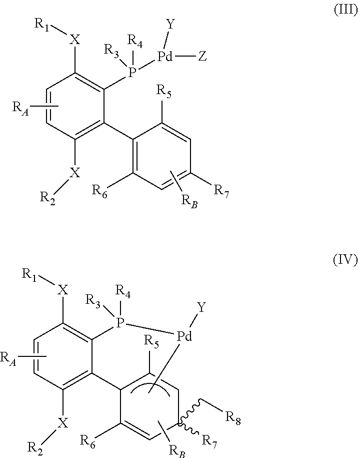
wherein
R1, R2 are same or different from each other and being linear or branched alkyl;
R3, R4, are same or different from each other and being branched or cyclic alkyl;
R5, R6, R7 are same or different from each other and being H, linear or branched or cyclic alkyl;
R8 is an alkenyl, aryl or aryl alkenyl group;
R_{A} and R_{B} are if present independently from each other one or more alkyl, cycloalkyl, aryl, heteroaryl, heteroalkyl, aralkyl, hydrogen, halogen, heteroaralkyl, alkoxyl, dialkylaminyl, trialkylsilyl; and
X is O or S, with O being preferred;
Y is an anionic ligand;
Z is a σ-donor ligand;
adding as a further reactant an aryl halide or heteroaryl halide to the reaction mixture; and
adding a base; and
isolating the arylhydrazine from the reaction mixture.

2. Process according to claim 1, wherein R1 is methyl, ethyl, propyl, iso-propyl, butyl, iso-butyl and sec-butyl, R2 is methyl, ethyl, propyl, iso-propyl, butyl, iso-butyl and sec-butyl, R3 is tert-butyl, isopropyl or 1-adamantyl, propellane, R4 is tert-butyl, isopropyl or 1-adamantyl, propellane; R5 is methyl, ethyl, propyl, isopropyl, butyl, iso-butyl and sec-butyl, R6 methyl, ethyl, propyl, isopropyl, butyl, iso-butyl and sec-butyl, R7 is methyl, ethyl, propyl, isopropyl, butyl, iso-butyl and sec-butyl.

3. Process according to claim 1, wherein the aryl halide is selected from the group consisting of phenyl, naphthyl, pyridyl, pyrazinyl and thienyl.

4. Process according to claim 1, wherein the organic solvent is selected from the group consisting of THF, dioxane, DME, CPME, 2-MeTHF.

5. Process according to claim 1, wherein the base is selected from the group consisting of KOH, K₂CO₃, K₃PO₄, Cs₂CO₃, NaOtBu, KOtBu, NaOMe, NaOH.

6. Process according to claim 1, wherein the process is conducted at a temperature of 10° C. to 80° C.

7. Process according to claim 1, wherein the arylhydrazine is isolated by acidified by concentrated hydrochloride acid to generate the hydrochloride salts and purified by two-phase extractions, precipitation and filtration.

8. Process according to claim 1, wherein Z is a bidentate ligand in which one part of the ligand is connected to the Palladium via a σ-donor bond as mentioned before and the other part is connected to the Palladium via a heteroatomic substituent.

9. Catalyst for the monoarylation of hydrazines of general formula (IV) 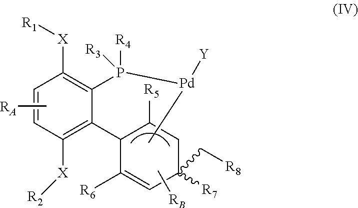
wherein R1, R2 are same or different from each other and being linear or branched alkyl;
R3, R4, are same or different from each other and being branched or cyclic alkyl;
R5, R6, R7 are same or different from each other and being H, linear or branched or cyclic alkyl;
R8 is an alkenyl, aryl or aryl alkenyl group;
R_{A} and R_{B} are if present independently from each other one or more alkyl, cycloalkyl, aryl, heteroaryl, heteroalkyl, aralkyl, hydrogen, halogen, heteroaralkyl, alkoxyl, dialkylaminyl, trialkylsilyl; and
X is O or S, with O being preferred;
Y is a an anionic ligand.

10. Process for the production of a compound according to claim 8, characterized in that
a compound of general formula (VI) 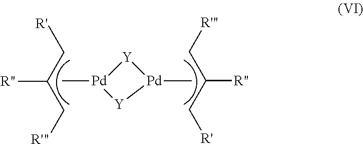
wherein R′, R″, R′″ are independently of each other selected from the group consisting of H, alkyl, aryl or R′ and R″ form an aromatic or non-aromatic cyclic ring;
Y is a halide;
and a ligand of formula (III) 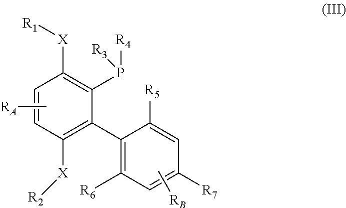
wherein
R1, R2 are same or different from each other and being linear or branched alkyl;
R3, R4, are same or different from each other and being branched or cyclic alkyl;
R5, R6, R7 are same or different from each other and being H, linear or branched or cyclic alkyl;
R_{A} and R_{B} are if present independently from each other one or more alkyl, cycloalkyl, aryl, heteroaryl, heteroalkyl, aralkyl, hydrogen, halogen, heteroaralkyl, alkoxyl, dialkylaminyl, trialkylsilyl;
X is O or S;
are reacted under conditions sufficient to produce the compound of the present claim.
